(19) United States
(12) Patent Application Publication
(10) Pub. No.: US 2019/0104991 A1
(43) Pub. Date: Apr. 11, 2019

(54) SELF CLOSING FINGER CUFF

(71) Applicant: Edwards Lifesciences Corporation, Irvine, CA (US)

(72) Inventors: Berend Westerhof, Amstelveen (NL); Olaf Schraa, Amsterdam (NL); Christianus Joannes Jozef Eugène van Groeningen, Utrecht (NL); Peiyuan Li, Amsterdam (NL)

(21) Appl. No.: 16/142,903

(22) Filed: Sep. 26, 2018

Related U.S. Application Data
(60) Provisional application No. 62/571,098, filed on Oct. 11, 2017.

(51) Int. Cl. A61B 5/00; A61B 5/022; A61B 5/0225

(52) U.S. Cl. CPC A61B 5/6831; A61B 5/6826; A61B 5/02255; A61B 5/02241

(57) ABSTRACT

Disclosed is a finger cuff that is attachable to a patient's finger to be used in measuring the patient's blood pressure by a blood pressure measurement system utilizing a volume clamp method. The finger cuff comprises a wrappable portion that wraps around a patient's finger. The wrappable portion includes a bladder and a light emitting diode (LED) and photodiode (PD) pair. The finger cuff further comprises a plurality of tubules mountable on an exterior of the wrappable portion. The tubules are inflatable to provide pressure to the wrappable portion of the finger cuff to the patient's finger and to provide tightness of the wrappable portion of the finger cuff to the patient's finger. When the finger cuff is placed around the patient's finger, the bladder and the LED-PD pair aid in measuring the patient's blood pressure by the blood pressure measurement system utilizing the volume clamp method.

### CROSS-REFERENCE TO RELATED APPLICATIONS

This application claims priority to U.S. Provisional Application No. 62/571,098, filed Oct. 11, 2017, the contents of which are incorporated herein by reference in their entirety.

### BACKGROUND

### Field

Embodiments of the invention relate generally to non-invasive blood pressure measurement. More particularly, embodiments of the invention relate to a finger cuff for blood pressure measurement.

### Relevant Background

Volume clamping is a technique for non-invasively measuring blood pressure in which pressure is applied to a patient's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged or wrapped around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in the measurement of the arterial volume. To accurately measure blood pressure, the feedback loop provides sufficient pressure generating and releasing capabilities to match the pressure oscillations of the patient's blood pressure.

Today, finger cuff based blood pressure monitoring devices generally use the same technology (e.g., photoplethysmography or similar technologies) to measure blood pressure. Unfortunately, such finger cuff devices may not be easily attachable to a patient's finger and may not be that accurate due to the finger cuff's positioning and snugness or tightness on the patient's finger.

### SUMMARY

Embodiments of the invention may relate to a finger cuff that is attachable to a patient's finger to be used in measuring the patient's blood pressure by a blood pressure measurement system utilizing a volume clamp method. The finger cuff comprises a wrappable portion that wraps around a patient's finger. The wrappable portion includes a bladder and a light emitting diode (LED) and photodiode (PD) pair. The finger cuff further comprises a plurality of tubules mountable on an exterior of the wrappable portion. The tubules are inflatable to provide pressure to the wrappable portion of the finger cuff to the patient's finger and to provide tightness of the wrappable portion of the finger cuff to the patient's finger. When the finger cuff is placed around the patient's finger, the bladder and the LED-PD pair aid in measuring the patient's blood pressure by the blood pressure measurement system utilizing the volume clamp method.

### BRIEF DESCRIPTION OF THE DRAWINGS

FIG. 1 is a diagram of an example of a blood pressure measurement system according to one embodiment.

FIG. 2 is a diagram illustrating an example of a conventional finger cuff.

FIGS. 3A-3C are diagrams illustrating the example of the conventional finger cuff that is arranged around a finger of a patient.

FIGS. 4A-4C are diagrams illustrating views of examples of finger cuffs according to embodiments of the invention.

FIG. 5 is a block diagram illustrating an example environment in which embodiments of the invention may be practiced.

### DETAILED DESCRIPTION

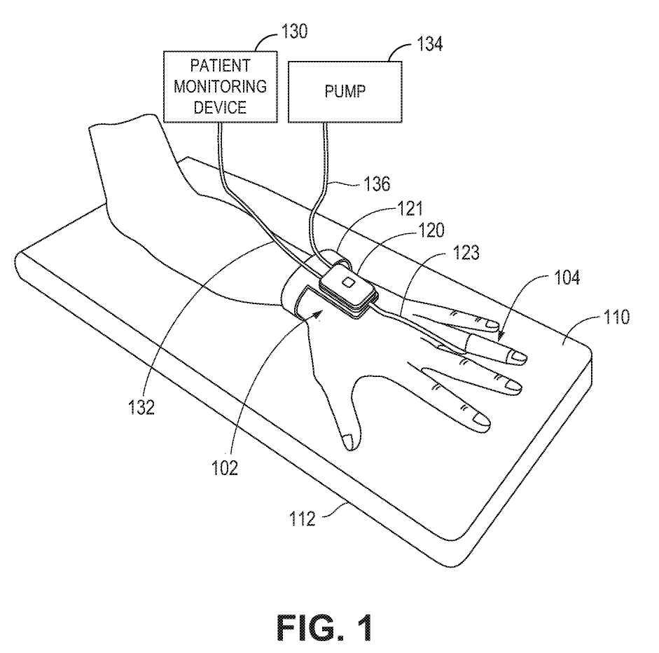

With reference to FIG. 1, which illustrates an example of a blood pressure measurement system according to one embodiment, a blood pressure measurement system 102 that includes a finger cuff 104 that may be attached to a patient's finger and a blood pressure measurement controller 120, which may be attached to the patient's body (e.g., a patient's wrist or hand) is shown.

The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 (e.g., to keep the pleth signal constant) and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some embodiments, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system can be used, including but not limited to the blood pressure measurement controller, and may be described simply as a pressure generating and regulating system that may be used with a finger cuff 104 including an LED-PD pair and a bladder to implement the volume clamping method.

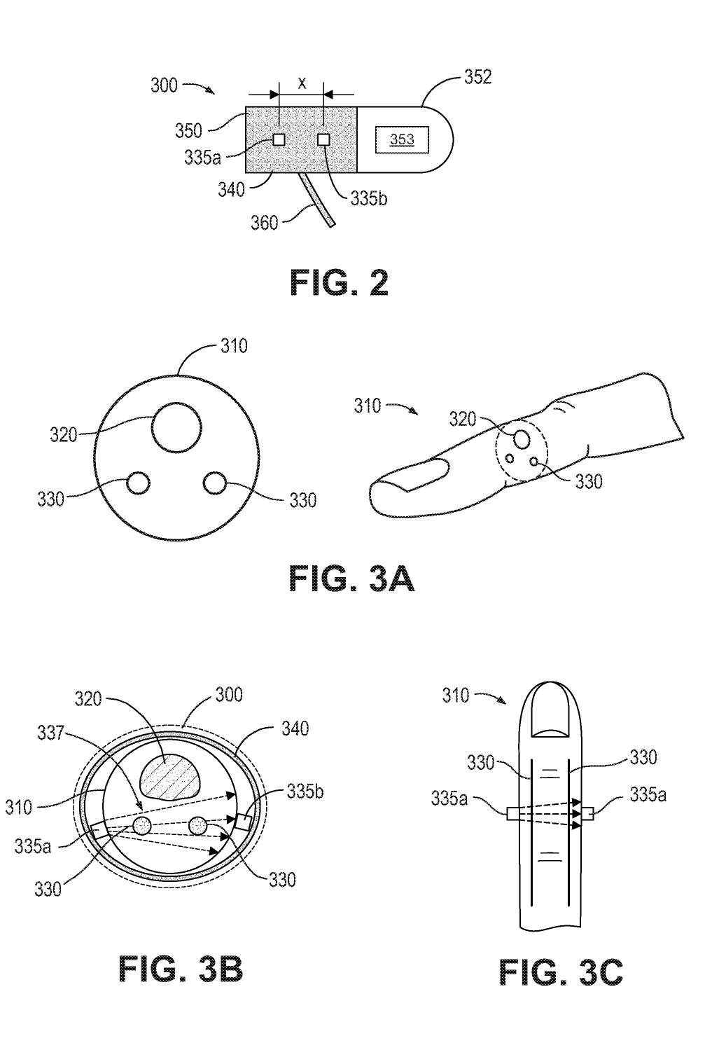

FIG. 2 is an example of a conventional finger cuff. With reference to FIG. 2, a conventional finger cuff 300 may be formed from a flexible material with a Velcro clamping system. The finger cuff 300 may include a first side 350 and a second side 352. In one embodiment, for attachment purposes to a patient's finger, the second side 352 on the interior may include a first connecting portion 353 (e.g., a Velcro type portion) that connects with a second connecting portion (not shown), e.g., a Velcro type portion, on the exterior of the first side 350 of the finger cuff 300. In another embodiment, the first connecting portion 353 may include removable or reusable adhesive material that can be removably attached to the exterior surface of the first side 350 of the finger cuff 300. It should be appreciated that this is just one example of an attachment mechanism and that any suitable type may be utilized (e.g., adhesive, tape, mechanical latching mechanisms, etc.). It should be appreciated that any type of wrappable finger cuff, fixed type of finger cuff, or any type of finger cuff may be used, and this is just one example.

Further, finger cuff 300 may include a bladder 340 and an LED-PD pair 335a-b mounted on the interior of the finger cuff 300. In one embodiment, the bladder 340 may include a pair of openings that surround the LED-PD pair 335a-b, respectively. The bladder 340 and LED-PD pair 335a-b may be coupled to tube or cable 360 through a connector, which may be attached to finger cuff 300, to provide pneumatic pressure to the bladder 340, and to provide power to and receive data from the LED-PD pair 335a-b. The LED-PD pair 335a-b may be used to perform measurements of a pleth signal to aid in measuring the patient's blood pressure.

With additional reference to FIGS. 3A-3C, the conventional finger cuff 300 may be wrapped around a patient's finger 310 that may include a finger bone 320 and one or more finger arteries 330. In operation, light (or optical signals) 337 generated by LED 335a may be transmitted or emitted in multiple directions through the finger 310, in which the finger arteries 330 are present. The PD 335b may detect some or all of the light 337 from the LED 335a. The amount of light registered by the PD 335b may be inversely proportional to the artery diameters of the finger arteries 330 and indicative of the pressure of the finger arteries 330.

For example, in FIGS. 3A-3C, the bladder 340 may be inflated to exert pressure on the finger arteries 330. If the pressure is high enough, for example, it may compress the finger arteries 330, thereby decreasing the diameter of the finger arteries 330 and increasing the amount of light registered by the PD 335b. Conversely, if the pressure is not high enough, it may compress the finger arteries 330 to a lesser extent (or may not compress at all), thereby increasing the diameter of the finger arteries 330 and decreasing the amount of light registered by the PD 335b. The amount of pressure necessary in the bladder 340 to compress the arteries 330 is dependent on the blood pressure. Therefore, by controlling the pressure of the inflatable bladder 340 such that the diameter of the finger arteries 330 is kept constant, the blood pressure of the patient may be computed and monitored as the pressure in the inflatable bladder 340 is directly linked to the blood pressure. As an example, as part of the volume clamp method, the pneumatic pressure is applied to the bladder 340 of the finger cuff based upon measuring the pleth signal received from the LED-PD pair 335a and 335b of the finger cuff (e.g., to keep the pleth signal constant) so that the pressure applied to the bladder 340 and measured by a pressure sensor should be correlated to the patient's blood pressure.

With respect to the conventional finger cuff 300, previously described, it is generally available in different sizes (e.g., small, medium, large), and includes differently sizes bladders 340 (depending on the size of the finger cuff 300). The distance between the LED 335a and the PD 335b (which may be referred to as “x”), therefore, may vary depending on the size of the finger cuff and bladder to meet the different finger sizes and finger physiology of the patient. In applying the finger cuff 300 on a patient's finger (e.g., finger 310), it is important for a healthcare provider to select a suitable finger cuff size for the patient such that the LED-PD pair 335a-b is properly and effectively positioned on the patient's finger in order to obtain an accurate optical measurement. However, the various sized conventional finger cuffs 300 may not be suitable for the finger sizes and physiology of many patients such that inadequate finger cuff attachments by health care providers may occur. In such circumstances, the LED-PD pair 335a-b may not be properly positioned on the patient's finger, and therefore, the PD 335b may not adequately detect or register light signals from the LED 335a (as shown in FIG. 3B), thereby producing a low quality signal (e.g., a pleth signal) that may result in an inaccurate blood pressure measurement.

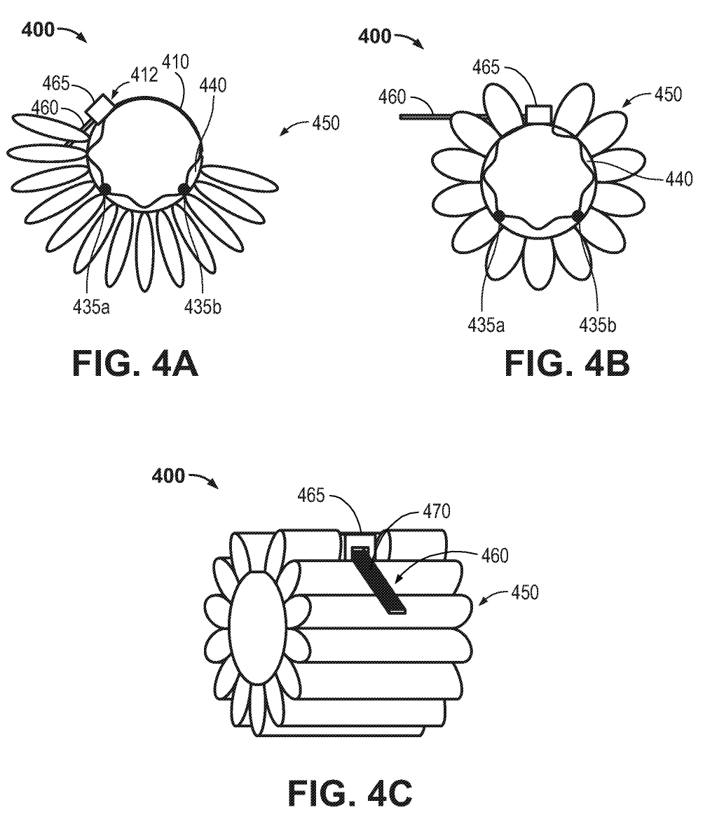

Embodiments shown in FIGS. 4A-4C, which are diagrams illustrating views of examples of a finger cuff 400 according to embodiments of the invention, may be utilized. Finger cuff 400 may mitigate or eliminate many of the problems associated with the convention finger cuff 300.

With reference to FIGS. 4A-4C, finger cuff 400 may be wrapped around a patient's finger 310 having finger bone 320 and finger arteries 330 (as previously described). As shown, finger cuff 400 may include a wrapping (or wrappable) portion 410 that wraps around the patient's finger. Wrapping portion 410 may include a first side and a second side, and may be formed of flexible material or may be a fixed finger cuff, as previously described.

In one embodiment, for attachment purposes to a patient's finger, finger cuff 400 may utilize a latching mechanism, for example a releasable tie wrap (or cable tie) fastening system (i.e., a tie wrap system that may be closed/locked in place and may thereafter be released), to latch the wrapping portion 410 to the patient's finger. For example, as shown, the first side of the wrapping portion 410 may include a head 465 and the second side of the wrapping portion 410 may include an elongated strap 460 (which may be formed of flexible material, e.g., nylon). The head 465 may include a strap entrance end 412 for insertion of the strap 460. Although not shown, in some embodiments, head 465 may include a locking device with a release tab. In some embodiments, as shown in FIG. 4C, strap 460 may include extending locking teeth 470 that are longitudinally spaced along the length of strap 460.

In operation, strap 460 may be pulled (e.g., by a healthcare provider) towards head 465 so that finger cuff 400 comfortably (or snuggly) fits around a patient's finger, and a tip of the strap 460 may be inserted through the strap entrance end 412. The locking device of head 465 may engage with a selected tooth 470 of the locking teeth so as to lock or secure the strap 460 in position within the head 465. Subsequently, to release strap 460 from head 465, the release tab may be activated to allow strap 460 to move in a release direction (e.g., a direction away from head 465), thereby permitting strap 460 to be removed or adjusted as desired.

Although a releasable tie wrap fastening system is illustrated in FIGS. 4A-4C, in some embodiments, finger cuff 400 may utilize any type of attachment mechanism (e.g., adhesive, tape, mechanical latching mechanisms, etc.) and finger cuff 400 may be any type of wrappable finger cuff, fixed type of finger cuff, or any type of finger cuff structure, as previously described.

As particularly shown in FIGS. 4A-4B, in one embodiment, finger cuff 400 may include a bladder 440 and an LED-PD pair 435a-b mounted on the interior of the wrapping portion 410. In one embodiment, bladder 440 may include openings that surround the LED-PD pair 435a-b. The bladder 440 and LED-PD pair 435a-b may be coupled to a tube or cable through a connector, which may be attached to finger cuff 400, to provide pneumatic pressure to the bladder 440, and to provide power to and receive data from the LED-PD pair 435a-b.

As further shown in FIGS. 4A-4C, finger cuff 400 may further include inflatable tubules 450 that are attached to (e.g., permanently attached or removably attached) or mounted on the exterior of the wrapping portion 410, and surround the wrapping portion 410. In one embodiment, tubules 450 may be approximately cylindrically-shaped and may include round edges. In one embodiment, tubules 450 may be longitudinally arranged along the length of the wrapping portion 410. In one embodiment, each of the tubules 450 may be attached to one another along the side of the tubule, or may be separately spaced from one another. It should be appreciated that these inflatable tubules are used as an example shape, and that any suitable shape, size, or structure to apply pressure to the finger cuff may be utilized.

Operationally, after the wrapping portion 410 is secured around a patient's finger (e.g., using the releasable tie wrap fastening system, as previously described), tubules 450 may be inflated (e.g., filled with a liquid or gas) to exert pressure and form tightness around the patient's finger so that finger cuff 400 effectively (or comfortably) fits around the patient's finger. Accordingly, the tightness or snugness of the finger cuff 400 around the patient's finger is dependent on the pressure provided to the tubules 450. Therefore, the tightness or snugness of the finger cuff 400 can be standardized by applying standardized pressure to the tubules 450 providing a snug or tight fit to the patient's finger improving the accuracy of the blood pressure measurement by the finger cuff.

With the previously described embodiments, the application of the finger cuff 400 to a patient's finger by a healthcare provider is less susceptible to application errors. Further, the finger cuff 400, with the adjustment ability of the latching mechanism and the pressure exerted by tubules 450, allows for the finger cuff 400 to be single-sized or a one-size-fits-all finger cuff, such that different finger cuff sizes (as previously discussed with respect to finger cuff 300) do not need to be utilized and a single-sized finger cuff 400 may be utilized for almost any size finger.

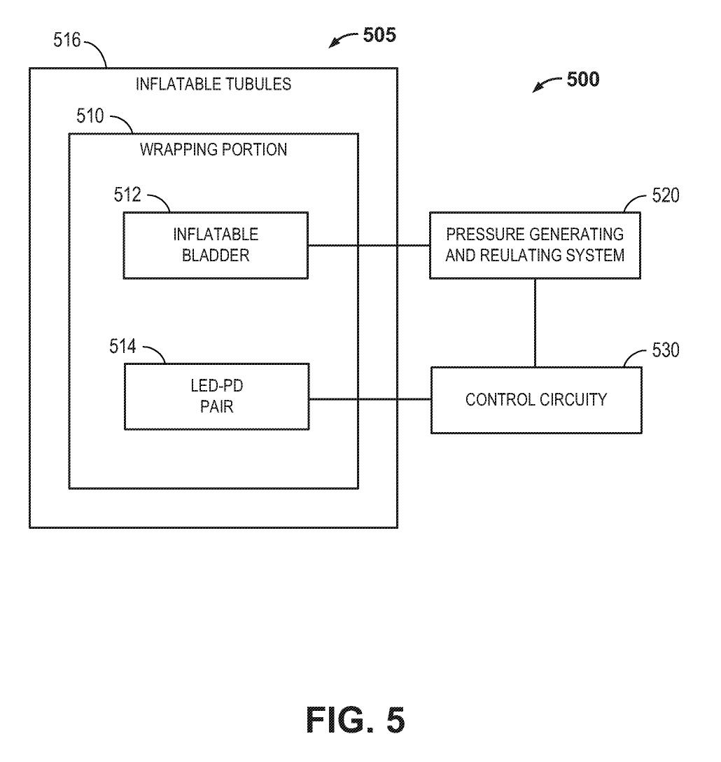

FIG. 5 is a block diagram illustrating an example environment 500 in which embodiments of the invention may be practiced. As shown in FIG. 5, finger cuff 505 may include wrapping portion 510, an inflatable bladder 512, an LED-PD pair 514, and inflatable tubules 516. The tubules 516 may be attached to or mounted on the exterior of the wrapping portion 510. The tubules 516 may be inflated or deflated (e.g., using a pump) to exert desired pressure so that finger cuff 505 may comfortably fit around a patient's finger. The inflatable bladder 512 may be pneumatically connected to a pressure generating and regulating system 520. The pressure generating and regulating system 520 and control circuitry 530 may generate, measure, and regulate pneumatic pressure that inflates or deflates the inflatable bladder 512 and/or tubule 516, and may further comprise such elements as a pump, a valve, a pressure sensor, and/or other suitable elements, as previously described. In particular, pressure generating and regulating system 520 in cooperation with control circuitry 530 may be configured to implement a volume clamp method with the finger cuff 510 by: applying pneumatic pressure to the inflatable bladder 512 of the finger cuff 505 to replicate the patient's blood pressure based upon measuring pleth signals received from the LED-PD pair 514 (e.g., to keep the pleth signal constant), and measuring the patient's blood pressure by monitoring the pressure of the inflatable bladder 512 based upon input from a pressure sensor, which should be the same or correlated to the patient's blood pressure, and may further command the display of the patient's blood pressure on the patient monitoring device.

It should be appreciated that aspects of the invention previously described may be implemented in conjunction with the execution of instructions by processors, circuitry, controllers, control circuitry, etc. As an example, control circuitry may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A finger cuff connectable to a patient's finger to be used in measuring the patient's blood pressure by a blood pressure measurement system utilizing the volume clamp method, the finger cuff comprising:
a wrappable portion that wraps around a patient's finger, the wrappable portion including a bladder and a light emitting diode (LED) and photodiode (PD) pair; and
a plurality of tubules mountable on an exterior of the wrappable portion, the tubules being inflatable to provide pressure to the wrappable portion of the finger cuff to the patient's finger and to provide tightness of the wrappable portion of the finger cuff to the patient's finger, and wherein, when the finger cuff is placed around the patient's finger, the bladder and the LED-PD pair aid in measuring the patient's blood pressure by the blood pressure measurement system utilizing the volume clamp method.

2. The finger cuff of claim 1, further comprising a latching mechanism to latch the wrappable portion of the finger cuff to the patient's finger.

3. The finger cuff of claim 2, wherein the latching mechanism includes a head and a strap.

4. The finger cuff of claim 3, wherein the strap is pulled towards, inserted through, and then secured within the head such that the wrappable portion of the finger cuff snuggly fits the patient's finger.

5. The finger cuff of claim 4, wherein the strap is releasable from the head such that the wrappable portion of the finger cuff can be removed from the patient's finger.

6. The finger cuff of claim 1, wherein the tubules are filled with a gas to provide pressure to the wrappable portion of the finger cuff to the patient's finger to provide tightness of the wrappable portion of the finger cuff to the patient's finger.

7. The finger cuff of claim 1, wherein the tubules are filled with a liquid to provide pressure to the wrappable portion of the finger cuff to the patient's finger to provide tightness of the wrappable portion of the finger cuff to the patient's finger.

8. A method to measure a patient's blood pressure by a blood pressure measurement system utilizing a finger cuff and a volume clamp method, the finger cuff comprising a wrappable portion including a bladder and a light emitting diode (LED) and photodiode (PD) pair and a plurality of tubules mountable on an exterior of the wrappable portion, the method comprising:
placing the finger cuff around a patient's finger such that the bladder and the LED-PD pairs aid in measuring the patient's blood pressure by the blood pressure measurement system utilizing the volume clamp method; and
inflating the tubules to provide pressure to the wrappable portion of the finger cuff to the patient's finger and to provide tightness of the wrappable portion of the finger cuff to the patient's finger.

9. The method of claim 8, further comprising latching, by a latching mechanism of the finger cuff, the wrappable portion of the finger cuff to the patient's finger.

10. The method of claim 9, wherein the latching mechanism includes a head and a strap.

11. The method of claim 10, wherein latching the wrappable portion of the finger cuff to the patient's finger includes pulling the strap towards the head, inserting the strap through the head, and securing the strap within the head such that the wrappable portion of the finger cuff snuggly fits the patient's finger.

12. The method of claim 11, further comprising releasing the strap from the head such that the wrappable portion of the finger cuff can be removed from the patient's finger.

13. A blood pressure measurement system utilizing a volume clamp method, the blood pressure measurement system comprising:
a finger cuff attachable to a patient's finger to be used in measuring the patient's blood pressure by the blood pressure measurement system utilizing the volume clamp method, the finger cuff comprising:
a wrappable portion that wraps around a patient's finger, the wrappable portion including a bladder and a light emitting diode (LED) and photodiode (PD) pair; and
a plurality of tubules mountable on an exterior of the wrappable portion, the tubules being inflatable to provide pressure to the wrappable portion of the finger cuff to the patient's finger and to provide tightness of the wrappable portion of the finger cuff to the patient's finger, and wherein, when the finger cuff is placed around the patient's finger, the bladder and the LED-PD pair aid in measuring the patient's blood pressure by the blood pressure measurement system utilizing the volume clamp method.

14. The blood pressure measurement system of claim 13, wherein the finger cuff further comprises a latching mechanism to latch the wrappable portion of the finger cuff to the patient's finger.

15. The blood pressure measurement system of claim 14, wherein the latching mechanism includes a head and a strap.

16. The blood pressure measurement system of claim 15, wherein the strap is pulled towards, inserted through, and then secured within the head such that the wrappable portion of the finger cuff snuggly fits the patient's finger.

17. The blood pressure measurement system of claim 16, wherein the strap is releasable from the head such that the wrappable portion of the finger cuff can be removed from the patient's finger.

18. The blood pressure measurement system of claim 13, wherein the tubules are filled with a gas to provide pressure to the wrappable portion of the finger cuff to the patient's finger to provide tightness of the wrappable portion of the finger cuff to the patient's finger.

19. The blood pressure measurement system of claim 13, wherein the tubules are filled with a liquid to provide pressure to the wrappable portion of the finger cuff to the patient's finger to provide tightness of the wrappable portion of the finger cuff to the patient's finger.
